(19) United States
(12) Patent Application Publication
(10) Pub. No.: US 2004/0247556 A1
(43) Pub. Date: Dec. 9, 2004

(54) Hair cosmetic preparations

(76) Inventors: Wilma M. Dausch, Limburgerhof (DE); Bernd de Potzolli, Bad Durheim (DE); Hideyuki Ichihara, Odawara Kanagawa (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/490,382

(22) PCT Filed: Oct. 1, 2002
(86) PCT No.: PCT/EP02/10984

(30) Foreign Application Priority Data
Oct. 12, 2001 (DE) 101 50 282.6

(51) Int. Cl. A61K 7/06; A61K 7/11

(52) U.S. Cl. 424/70.15; 424/70.17

(57) ABSTRACT

Hair-setting compositions comprising

a homopolymer of N-vinylcaprolactam or an anionic or nonionic copolymer of at least 70% by weight of N-vinylcaprolactam (polymer A) and

polymer B chosen from the group consisting of polyamides, polyurethanes, homo- and copolymers of monoolefinically unsaturated monomers and

panthenol in an amount of from 0.01 to 0.75% by weight.

The present invention relates to compositions which comprise polymers based on N-vinylcaprolactam. Hairspray compositions which consist of a polyester and a water-soluble polymer, for example a copolymer of N-vinylcaprolactam and N-vinylpyrrolidone, are known from WO 93/17658. However, such hairsprays are hydrolysis-sensitive and can therefore only be used to a limited extent in the application and handling.

EP 0 734 717 A2 describes hair-setting compositions comprising

A) 0.5 to 20% by weight of a nonionic or anionic homo- or copolymer of at least 70% by weight of N-vinylcaprolactam (polymer A), and

B) 0.5 to 20% by weight of a further film-forming polymer (polymer B) chosen from the group consisting of polyamides, polyurethanes, homo- and copolymers of monoolefinically unsaturated monomers.

It is an object of the present invention to develop hair-setting compositions which have good setting properties coupled with good wash-out and low stickiness; furthermore, the hair-setting compositions should have good hold with simultaneously good curl retention.

We have found that this object is achieved by hair-setting compositions which comprise

a homopolymer of N-vinylcaprolactam or an anionic or nonionic copolymer of at least 70% by weight of N-vinylcaprolactam (polymer A) and

polymer B chosen from the group consisting of polyamides, polyurethanes, homo- and copolymers of monoolefinically unsaturated monomers and

panthenol in an amount of from 0.01 to 0.75% by weight.

We have found that compositions which comprise a homopolymer of N-vinylcaprolactam or an anionic or a nonionic copolymer of at least 70% by weight of N-vinylcaprolactam and panthenol in an amount of from 0.1 to 0.75% by weight increases the setting action in hair cosmetic preparations and simultaneously have good curl retention.

Suitable polymers A are homopolymers of N-vinylcaprolactam, which are known to the person skilled in the art and can be prepared, for example, in accordance with the procedure described in U.S. Pat. No. 3,145,147.

Further suitable polymers A are copolymers of N-vinylcaprolactam and further polymerizable monomers, where the copolymers consist of at least 70% by weight, preferably at least 85% by weight, of vinylcaprolactam.

Further suitable polymerizable monomers for the copolymers (A) are:

monomers with an acid function, such as acrylic acid, methacrylic acid, acrylamidomethylpropylsulfonic acid (AMPS), 3-sulfopropyl (meth)acrylate, optionally also in completely or partially neutralized form;

C₁-C₁₈-alkyl (meth)acrylates, such as tert-butyl acrylate, ethyl acrylate, isobutyl methacrylate, n-butyl methacrylate, methyl methacrylate, ethyl methacrylate and hydroxyalkyl (meth)acrylate; vinyl esters of C₂-C₁₀-fatty acids, such as vinyl acetate, vinyl propionate and vinyl esters of longer-chain and/or branched fatty acids, for example versatic acid;

C₃-C₈-N-alkyl (meth)acrylamides, such as methacrylamide, N,N-dimethylacrylamide, N-tert-butylacrylamide and N-tert-octylacrylamide; N-vinylpyrrolidone and N-vinylpiperidone.

The further monomers can be used as an individual compound or as a mixture for the copolymer (A).

Such copolymers are known or can be prepared by customary polymerization processes.

For example, EP 455 081 describes copolymers of N-vinylpyrrolidone, N-vinylcaprolactam and N-vinylimidazole. EP 74 191 discloses copolymers of N-vinylpyrrolidone, N-vinylcaprolactam and dimethylaminoethyl methacrylate.

For the hair cosmetic preparations, preference is given to using homopolymers of N-vinylcaprolactam. These are obtainable, for example, under the trade name Luviskol® Plus (BASF Aktiengesellschaft).

The hair cosmetic preparations comprise the polymers A in an amount of from 0.1 to 25% by weight, preferably from 0.1 to 10% by weight, in particular 0.3 to 5% by weight, based on the finished composition.

Panthenol can be used as D-panthenol or as a racemic mixture D,L-panthenol.

Particularly suitable compositions are those which comprise 0.01 to 0.75% by weight of panthenol, preferably 0.01 to 0.5% by weight, particularly preferably 0.1 to 0.3% by weight.

In a preferred embodiment, the hair cosmetic preparations comprise

0.1 to 25% by weight, in particular 0.1 to 10% by weight, preferably 0.3 to 5% by weight, of polymer A and

0.1 to 25% by weight, in particular 1 to 10% by weight, preferably 1 to 5% by weight, of polymer B

0.01 to 0.75% by weight, in particular 0.01% by weight to 0.5% by weight, in particular 0.1 to 0.3% by weight, of panthenol.

According to the invention, suitable polymers B are polymers different from A chosen from the group consisting of polyamides, polyurethanes and homo- and copolymers of monoolefinically unsaturated monomers. According to the invention film-forming polymers may be customary hair-setting polymers, but may also be film formers for industrial coatings and binders.

Suitable homo- and copolymers of monoolefinically unsaturated monomers are primarily homo- and copolymers of C₁-C₁₂-alkyl acrylates and methacrylates, monoolefinically unsaturated C₃-C₅-monocarboxylic acids and vinyl esters thereof, maleic acid and half-esters thereof, methyl vinyl ether, acrylamides, methacrylamides, N-alkyl-substituted (meth)acrylamides, N-vinylpyrrolidone, N-vinylimidazole, and styrene.

Particularly preferred polymers B are acrylate polymers, such as copolymers of acrylic acid/ethyl acrylate/N-tert-butylacrylamide, copolymers of methacrylic acid/tert-butyl acrylate/N-vinylpyrrolidone, copolymers based on acrylic acid and vinyl acetate, copolymers of octylacrylamide/acrylate/butylaminoethyl methacrylate, copolymers of octylacrylamide/acrylate, copolymers of ethyl ethacrylate/methacrylic acid/N-vinylpyrrolidone or copolymers of acrylate and hydroxyalkyl acrylate. Such copolymers are sold, for example, under the trade names Amerhold®, Ultrahold®8, Ultrahold Strong®, Luviflex®VBM, Luvimer® 100P, Luvimer®36D, Luvimer®30E, Luvimer®MAE, Acronal®500D, Stepanhold®, Luviflex® Soft, Lovocryl®, Versatyl®, Amphomer®, Acydyne® 258.

Preferred polymers B are copolymers of methacrylic acid/tert-butyl acrylate/dimethicone copolyol, as are available, for example, under the trade name Luviflex® Silk. These polymers and their preparation are described in WO 99/04750.

Preferred polymers B are sulfonate-containing polyamides consisting of:

20 to 99 mol % of a monoaminocarboxylic acid having 2 to 12 carbon atoms or lactam thereof,

0.5 to 40 mol % of a diamine having 2 to 18 carbon atoms,

0.5 to 25 mol % of a sulfonate-carrying dicarboxylic acid having 4 to 12 carbon atoms, and

Further preferred polymers B are sulfonate-carrying acrylates consisting of:

50 to 90% by weight of one or more monomers from the group consisting of C₁-C₁₈-alkyl esters of acrylic acid or of methacrylic acid and vinyl esters of saturated C₂-C₁₀-monocarboxylic acids,

10 to 25% by weight of a sulfonic acid-containing vinyl monomer, and

0 to 40% by weight of a further monomer from the group consisting of acrylic acid, methacrylic acid, acrylamide, methacrylamide, C₃-C₈-N-alkylacrylamide, N,N-dimethylacrylamide.

Likewise preferred polymers B are copolymers in the form of microdispersions according to DE-A 43 27 514, which are obtainable by free-radically initiated copolymerization or copolymerization initiated using ionizing radiation of

40 to 99% by weight of one or more water-insoluble, monoethylenically unsaturated monomers,

1 to 60% by weight of one or more water-soluble monoethylenically unsaturated monomers and

0 to 30% by weight of one or more polyethylenically unsaturated monomers in aqueous medium in the presence of from 2 to 20% by weight, based on the total amount of monomers, of surface-active compounds as emulsifier, having an average particle size of from 5 to 37 nm, determined by light scattering in aqueous medium.

Suitable polymers B are also homo- and copolymers of N-vinylpyrrolidone, as are sold, for example, under the name Luviskol® by BASF Aktiengesellschaft. The copolymers are obtainable by polymerization of N-vinylpyrrolidone with vinyl acetate and/or vinyl propionate in various weight ratios. Examples of such polymers are:

Luviskol®K17, Luviskol®K30, Luviskol®K60, Luviskol®K80, Luviskol®K90 (polyvinylpyrrolidones with a corresponding K value as powder or as solution (aqueous or aqueous/alcoholic).

Luviskol®VA=vinylpyrrolidone/vinyl acetate copolymers, in particular Luviskol®VA 73, Luviskol®VA 64, Luviskol®VA 55, Luviskol®VA 37, Luviskol®VA 28.

Further suitable polymers B are ternary polymers of vinylpyrrolidone, vinyl acetate and vinyl propionate, for example Luviskol®VAP 343.

Likewise suitable are vinyl acetate copolymers with crotonic acid, for example the products Luviset®CA, Luviset®CAP, Luviset®CAN, Resyn® 28-2930 and Resyn® 28-2942.

It is also possible to use quaternized polyquaternium-4, polyquaternium-6, polyquaternium-7., polyquaternium-10, polyquaternium-11 or polyquaternium 16.

Further suitable polymers B are copolymers of methyl vinyl ether (MVE) and maleates, for example copolymers of MVE/monoethyl maleate, MVE/monoisopropyl maleate or MVE/monobutyl maleate, for example the products Gantrez ES-225, Gantrez®ES-335, Gantrez®ES-425, Gantrez®ES-435, Gantrez®SP-215, Gantrez® Super A 420.

It is also possible to use polyurethanes as are known, for example, from WO 94/03510 as polymer B. Likewise suitable are sodium salts of polystyrenesulfonic acid, as are sold under the name Flexan®130.

Further polymers B are copolymers as described in WO 00/11051.

The polymers A and B can, if they are constructed from acid-containing monomers, be used in the acid form or in partially or completely neutralized form for the hair cosmetic preparations. For the neutralization, alkali metal hydroxides, ammonia, organic amines, in particular amino alcohols, are suitable, very particular preference being given to 2-amino-2-methyl-1-propanol.

The polymers A and B are usually present in a solvent, the solvent used preferably being water or alcohols or mixtures of water and lower alcohols. The proportion of solvent in the hair-setting composition is usually between 25 and 98% by weight.

The mixing together of the polymers A and B can take place by dissolving A and B as powders in a solvent, or by mixing one polymer as a solid or as a solution into a solution of the other polymer. Polymer B can also be in the form of a dispersion, it being possible to add polymer A as powder or aqueous or aqueous/alcoholic solution.

The hair cosmetic preparations comprise the polymers B in an amount of from 0.1 to 25% by weight, in particular 0.5 to 20.0% by weight, preferably 1 to 10% by weight, in particular 1 to 5% by weight, based on the finished composition.

In addition to the polymers A, panthenol and B, and a solvent, the hair cosmetic preparations can comprise, depending on their intended use, further customary hair cosmetic additives, such as perfume oils, emulsifiers, preservatives, care substances such as collagen, vitamins, protein hydrolyzates, stabilizers, pH regulators, dyes and further customary additives.

If the hair cosmetic preparations are to be used as hairspray, then a propellant is usually added. Customary propellants are lower alkanes, for example propane or butane, dimethyl ether, nitrogen, nitrous oxide or carbon dioxide or mixtures of these substances. The hair-setting compositions according to the invention can also comprise halogen-containing hydrocarbons as propellant. If mechanical spray devices, for example spray pumps, are used, the propellant can be dispensed with.

The hair cosmetic preparations have excellent performance properties; they form clear films and, in aqueous/alcoholic solutions, have a low solution viscosity, meaning that even in relatively high concentrations they still have good spray properties. Surprisingly, even water-insoluble film-forming polymers in combination with polyvinylcaprolactams can be processed to give hair-setting compositions which can be readily washed out. The hair-cosmetic preparations surprisingly have good hold and simultaneously good curl retention.

The polyvinylcaprolactam solutions used in the examples below were 40% strength by weight solutions in ethanol. The K value according to Fikentscher of the polymer was 40 (1% in ethanol).

Performance Investigations

The values in the flexural test were measured at 20° C. and 65% relative humidity in cN.

For this, in each case 3% of polymer B, optionally 100% neutralized with AMP in ethanol abs., were measured together with polymer A (Luviskol®Plus) and panthenol USP.

Polymer B

Ultrahold® Strong pulverulent terpolymer of N-tertiary-butylacrylamide, ethyl acrylate, acrylic acid

Luvimer® 100 P powder of a copolymer of tertiary-butyl acrylate/ethyl acrylate/methacrylic acid

Luviflex® Silk terpolymer of tert-butyl acrylate/methacrylic acid/dimethicone copolyol; 50% strength ethanolic solution

Luviset® P.U.R. polyurethane according to Example 3, WO 94/03510 as 30% strength by weight aqueous microdispersion

Luviskol® Plus 40% strength by weight ethanolic solution of polyvinylcaprolactam

(i.e. a 2.5% strength by weight solution of Luviskol® Plus corresponds to 1% by weight of polyvinylcaprolactam)

| | | | | | | Comparison |
|---|---|---|---|---|---|---|
| | | | | with 0.5% | | with 1% |
| | | | | panthenol USP | Comparison | panthenol USP |
| | | Without | Comparison | and 2.5% Luviskol | with 2.5% | and 2.5% |
| | | addition | with 0.5% | Plus | Luviskol Plus | Luviskol Plus |
| | | in % | panthenol USP | (solution) | (solution) | (solution) |
| A1 | Ultrahold Strong | 146 | 131 | 176 | 148 | 204 |
| B1 | Luvimer 100 P | 120 | 146 | 192 | 150 | 165 |
| C1 | Luviflex Silk | 111 | 103 | 183 | 175 | 161 |
| D1 | Luviset P.U.R. | 157 | 155 | 216 | 187 | 210 |

The curl retention was measured at 30° C. and 90% relative humidity. For this, in each case 3% of polymer B, optionally 100% neutralized with AMP in ethanol abs., and 40% propane/butane was formulated.

Sample A 4 was formulated with 40% DME (dimethyl ether) instead of propane/butane.

| | | | | | | Comparison |
|---|---|---|---|---|---|---|
| | | | | with 0.5% | | with 1% |
| | | | | panthenol USP | Comparison | panthenol USP |
| | | Without | Comparison | and 2.5% Luviskol | with 2.5% | and 2.5% |
| | | addition | with 0.5% | Plus | Luviskol Plus | Luviskol Plus |
| | | in % | panthenol USP | (solution) | (solution) | (solution) |
| A1 | Ultrahold Strong | 90 | 50 | 86 | 96 | 67 |
| A2 | Luvimer 100 P | 88 | 72 | 85 | 94 | 71 |
| A3 | Luviflex Silk | 81 | 63 | 75 | 94 | 50 |
| A4 | Luviset P.U.R. | 83 | 38 | 81 | 93 | 45 |

The tables show that for an amount of 0.5% panthenol (comparative example) on its own the curl retention is greatly reduced. Only with the preparations according to the invention is the desired hold with simultaneously high curl retention achieved.

The polyvinylcaprolactam solutions used in the examples below were 40% strength by weight solutions in ethanol.

### EXAMPLE 1

2.5% by wt. polyvinylcaprolactam solution

0.5% by wt. panthenol

10.0% by wt. polyurethane according to Example 3, WO 84/03510 as 25% strength by weight aqueous microdispersion

50.0% by wt. ethanol

ad 100 Aqua dist.

### EXAMPLE 2

2.5% by wt. polyvinylcaprolactam solution

0.5% by wt. panthenol USP

2.5% by wt. polyamide of 20 mol % ε-caprolactam, 34 mol % of hexamethylenediamine, 17 mol % of 5-sulfoisophthalic acid sodium salt and 17 mol % of isophthalic acid

50.0% by wt. ethanol

ad 100 Aqua dist.

### EXAMPLE 3

2.5% by wt. polyvinylcaprolactam solution

0.5% by wt. panthenol USP

10.0% by wt. acrylate microdispersion according to Example 10, DE-A 43 27 514 (solids content 20% by weight)

50.0% by wt. ethanol

ad 100 Aqua dist.

### EXAMPLE 4

2.5% by wt. polyvinylcaprolactam solution

0.5% by wt. panthenol USP

2.5% by wt. Luviskol® VA 64P, pulverulent copolymer of N-vinylpyrrolidone/vinyl acetate

52.5% by wt. ethanol abs.

ad 100 Aqua dist.

### EXAMPLE 5

2.5% by wt. polyvinylcaprolactam solution

0.5% by wt. panthenol USP

2.5% by wt. Luvimer® 100P, powder of a copolymer of tert-butyl acrylate/ethyl acrylate/methacrylic acid

0.58% by wt. 2-amino-2-methylpropanol (AMP)

52.50% by wt. ethanol abs.

ad 100 Aqua dist.

### EXAMPLE 6

2.5% by wt. polyvinylcaprolactam solution

0.5% by wt. panthenol USP

2.5% by wt. Luviset® CAP, pulverulent terpolymer of vinyl acetate/vinyl propionate/crotonic acid

0.19% by wt. AMP

52.50% by wt. ethanol abs.

ad 100 Aqua dist.

### EXAMPLE 7

2.5% by wt. polyvinylcaprolactam solution

0.5% by wt. panthenol USP

2.5% by wt. Ultrahold®8, pulverulent terpolymer of N-tert-butylacrylamide, ethyl acrylate, acrylic acid

0.23% by wt. AMP

52.50% by wt. ethanol abs.

ad 100 Aqua dist.

### EXAMPLE 8

2.5% by wt. polyvinylcaprolactam solution

0.5% by wt. panthenol USP

2.5% by wt. Amphomer®, alkylacrylamide/acrylate copolymer

0.41% by wt. AMP

52.50% by wt. ethanol abs.

ad 100 Aqua dist.

### EXAMPLE 9

2.5% by wt. polyvinylcaprolactam solution

0.5% by wt. panthenol USP

3.4% by wt. Acronal® 500D, 50% strength by weight aqueous anionic copolymer dispersion of an acrylate/vinyl acetate copolymer

50.0% by wt. ethanol

ad 100 Aqua dist.

### EXAMPLE 10

2.5% by wt. polyvinylcaprolactam solution

0.5% by wt. panthenol USP

2.5% by wt. Luvimer® loop

0.58% by wt. AMP

52.50% by wt. ethanol

ad 100 mixture of propane/butane in the ratio 50/50

### EXAMPLE 11

2.5% by wt. polyvinylcaprolactam solution

0.5% by wt. panthenol USP

2.5% by wt. Ultrahold® 8

0.23% by wt. AMP

32.27% by wt. ethanol

ad 100 mixture of propane/butane/n-pentane in the ratio 30/50/20

### EXAMPLE 12

1.25% by wt. polyvinylcaprolactam solution

0.5% by wt. panthenol USP

3.0% by wt. Luvimer® loop, powder of a copolymer of tert-butyl acrylate/ethyl acrylate/methacrylic acid

0.72% by wt. AMP

40.0% by wt. propane/butane 3.5

ad 100 ethanol abs.

### EXAMPLE 13

1.25% by wt. polyvinylcaprolactam solution

0.5% by wt. panthenol USP

3.0% by wt. Ultrahold® Strong, pulverulent terpolymer of N-tert-butylacrylamide, ethyl acrylate, acrylic acid

0.35% by wt. AMP

40.0% by wt. propane/butane 3.5

ad 100 ethanol abs.

### EXAMPLE 14

1.25% by wt. polyvinylcaprolactam solution

0.5% by wt. panthenol USP

6.0% by wt. Luviflex® Silk, terpolymer of tert-butyl acrylate/methacrylic acid/dimethicone copolyol; 50% strength ethanolic solution

0.73% by wt. AMP

ad 100 ethanol abs.

### EXAMPLE 15

1.25% by wt. polyvinylcaprolactam solution

0.3% by wt. panthenol USP

10.0% by wt. Luviset® P.U.R., polyurethane according to Example 1, WO 94/03510; 30% by weight aqueous dispersion)

40.0% by wt. propane/butane 3.5

ad 100 ethanol abs.

### EXAMPLE 16

No Rinse Foam Conditioner

A 81.5% by wt. Aqua

0.5% by wt. D-panthenol (Panthenol)

0.5% by wt. Luviquat® Mono CP (Hydroxyethyl Cetyldimonium Phosphate)

2.5% by wt. Luviquate HM 552 (Polyquaternium-16)

0.2% by wt. Cremophor®A 25 (Ceteareth-25)

2.0% by wt. Luviskol® Plus (Polyvinylcaprolactam)

B 0.3% by wt. Cremophor® RH 40 (PEG-40 hydrogenated castor oil)

qs fragrance/preservative

C 10.0% by wt. Propellant A46 (Isobutane & Propane)

### EXAMPLE 17

Styling Mousse Containing Luviflex® Soft and Luviskol® Plus

A 2.00% by wt. Luviquat Mono LS (Cocotrimonium Methosulfate)

qs Perfume oil

B 69.00% by wt. water demin. (Aqua demin.)

0.60% by wt. AMP (Aminomethyl Propanol)

6.70% by wt. Luviflex® Soft (Acrylates Copolymer)

2.50% by wt. Luviskol® Plus (Polyvinylcaprolactam)

0.20% by wt. Cremophor® A 25 (Ceteareth-25)

0.20% by wt. D-Panthenol® USP (Panthenol)

0.10% by wt. Unvinul® P 25 (PEG-25 PABA)

0.20% by wt. Natrosole 250 HR (Hydroxyethylcellulose)

8.50% by wt. ethanol 96% (Alcohol)

C 10.00% by wt. propane/butane 3.5 bar (20° C.)

### EXAMPLE 18

1.25% by wt. polyvinylcaprolactam solution

0.5% by wt. panthenol USP

6.0% by wt. of the polymer according to Example 22 of WO 00/11051

0.73% by wt. AMP

ad 100 ethanol abs.

1. A hair-setting composition comprising:
a homopolymer of N-vinylcaprolactam or an anionic or nonionic copolymer of at least 70% by weight of N-vinylcaprolactam (polymer A);
a polymer B is selected from the group consisting of a polyamide, a polyurethane, a homopolymer of a monolefinically unsaturated olefin, and a copolymer of monoolefinically unsaturated monomers; and
panthenol in an amount of from 0.01 to 0.75% by weight.

2. A hair-setting composition as claimed in claim 1, wherein panthenol is used in an amount of from 0.1 to 0.5% by weight.

3. A hair-setting composition as claimed in claim 1, wherein a homopolymer of N-vinylcaprolactam is used as polymer A.

4. A hair-setting composition as claimed in claim 1, wherein the polymer A is used in an amount of from 0.1 to 25% by weight.

5-9. (Cancelled).

10. A hairspray, which comprises:
the hair-setting composition as claimed in claim 1 and
a propellant.

11. A hairspray as claimed in claim 10, wherein the propellant is selected from the group consisting of propane, butane, a halogenated hydrocarbon, dimethylether, nitrogen, nitrous oxide, carbon dioxide, and mixtures thereof.

12. A hairspray, which comprises:
a homopolymer of N-vinylcaprolactam or an anionic or nonionic copolymer of at least 70% by weight of N-vinylcaprolactam (polymer A),
panthenol, and
a propellant.

13. A hairspray as claimed in claim 12, wherein the propellant is selected from the group consisting of propane, butane, a halogenated hydrocarbon, dimethylether, nitrogen, nitrous oxide, carbon dioxide, and mixtures thereof.

14. A hairspray as claimed in claim 12, which further comprises a polymer B selected from the group consisting of a polyamide, a polyurethane, a homopolymer of a monolefinically unsaturated monomer, and a copolymer of monoolefinically unsaturated monomers.

15. A hairspray as claimed in claim 12, wherein a homopolymer of N-vinylcaprolactam is used as polymer A.

16. A hairspray as claimed in claim 12, wherein the polymer A is present in an amount of from 0.1 to 25% by weight.

17. A hairspray as claimed in claim 12, wherein the polymer A is present in an amount of from 0.1 to 10% by weight.

18. A hairspray as claimed in claim 12, wherein the polymer A is present in an amount of from 0.3 to 5% by weight

19. A hairspray as claimed in claim 12, wherein panthenol is present in an amount of from 0.01 to 0.75% by weight, in particular 0.01 to 0.5% by weight.

20. A hairspray as claimed in claim 12, wherein panthenol is present in an amount of from 0.01 to 0.5% by weight.

21. A method for forming and/or maintaining a head of hair, which comprises:
applying the hairspray of claim 10 to hair.

22. A method for forming and/or maintaining a head of hair, which comprises:
applying the hairspray of claim 12 to hair.
